(19) United States
(12) Patent Application Publication
(10) Pub. No.: US 2025/0000773 A1
(43) Pub. Date: Jan. 2, 2025

(54) COMPOSITION COMPRISING GLYCOLIPID, TAURATE SURFACTANT, AND AMINO ACID SURFACTANT

(71) Applicant: L'OREAL, PARIS (FR)

(72) Inventors: Nodoka MITAKE, Kawasaki-shi (JP); Hiroaki KAGA, Kawasaki-shi (JP); Sho WATANABE, Kawasaki-shi (JP); Masanori ORITA, Kawasaki-shi (JP)

(21) Appl. No.: 18/810,852

(22) Filed: Aug. 21, 2024

Related U.S. Application Data
(63) Continuation of application No. PCT/JP2024/080083, filed on Jun. 7, 2024.

(30) Foreign Application Priority Data
Jun. 20, 2023 (JP) 2023-101048
Jul. 21, 2023 (FR) 2307851

(51) Int. Cl. A61K 8/60; A61K 8/44; A61K 8/46; A61Q 19/10

(52) U.S. Cl. CPC A61K 8/602; A61K 8/44; A61K 8/466; A61Q 19/10

(57) ABSTRACT

A composition contains: (a) at least one glycolipid; (b) at least one taurate surfactant in an amount of at least 0.6% by weight relative to the total weight of the composition; (c) at least one amino acid surfactant; and (d) water. The composition can be used as a cosmetic composition with an improved stability. In one embodiment, the amount of the (a) glycolipid(s) ranges from 0.5% to 20% by weight, preferably from 1% to 15% by weight, and more preferably from 2% to 10% by weight, relative to the total weight of the cosmetic composition.

### CROSS REFERENCE TO RELATED APPLICATION

This Application is a continuation of PCT/JP2024/080083 filed on Jun. 7, 2024 which, in turn, claims the priority of Japanese Patent Application No. 2023-101048 filed on Jun. 20, 2023 and French Patent Application No. 2307851 filed on Jul. 21, 2023, all of which are incorporated herein by reference.

### TECHNICAL FIELD

The present invention relates to a composition comprising at least one glycolipid, at least one taurate surfactant, and at least one amino acid surfactant, preferably a cosmetic composition comprising the same, and more preferably a rinse-off cosmetic composition comprising the same.

### BACKGROUND ART

The formulation of environmentally-friendly cosmetic products, which are designed and developed considering environmental issues, is becoming a major goal in an effort to meet global challenges.

It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin. 30

Biosurfactants like glycolipids such as rhamnolipids are materials of natural origin and are known to have unique bioactivities, such as anti-inflammation efficacy, anti-allergic efficacy, antibacterial efficacy, and the like. For example, glycolipids are known to have anti-inflammation efficacy and are potentially used on keratinous substances, such as skin.

For example, JP-T-2015-507626 discloses a composition comprising water, at least one biosurfactant and at least one fatty acid, wherein the fraction of the sum of all surfactants in the composition is from 1 to 30% by weight, and that the fraction of fatty acid, based on the sum of fatty acid and surfactants, is from 0.1 to 20% by weight. 40

However, although glycolipids have several unique bioactivities, there is a problem that composition comprising glycolipids may be less stable when the temperature changes.

Therefore, there is a need to provide a composition comprising glycolipids which exhibits an improved stability against a temperature change.

### DISCLOSURE OF INVENTION

An objective of the present invention is to provide a composition comprising at least one glycolipid, which has an improved stability against a temperature change.

The above objective of the present invention can be achieved by a composition comprising:
(a) at least one glycolipid;
(b) at least one taurate surfactant in an amount of at least 0.6% by weight relative to the total weight of the composition;
(c) at least one amino acid surfactant; and
(d) water.

The (a) glycolipid may be selected from rhamnolipids.

The amount of the (a) glycolipid(s) may range from 0.5% to 20% by weight, preferably from 1% to 15% by weight, and more preferably from 2% to 10% by weight, relative to the total weight of the cosmetic composition.

The (b) taurate surfactant may be represented by formula (I):

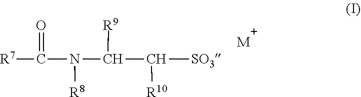

wherein
R⁷ is (C₈-C₂₂) alkyl;
R⁸ is H or (C₁-C₄) alkyl;
R⁹ and R¹⁰ are each independently H or (C₁-C₄) alkyl; and
M⁺ is a sodium, potassium, or ammonium cation.

The amount of the (b) taurate surfactant(s) may range from 0.6% to 10% by weight, preferably from 0.75% to 5% by weight, and more preferably from 0.9% to 3% by weight, relative to the total weight of the composition.

The the (c) amino acid surfactant may be represented by formula (A):

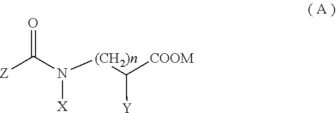

wherein:

Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and
M is a salt-forming cation wherein COO is the counter-anion, such as for example sodium, potassium, ammonium, or triethanolamine.

The amount of the (c) amino acid surfactant(s) may range from 0.5% to 20% by weight, preferably from 1% to 15% by weight, and more preferably from 2% to 10% by weight, relative to the total weight of the composition.

The amount of the (d) water may range from 50% to 99% by weight, preferably from 60% to 97% by weight, and more preferably from 70% to 95% by weight, relative to the total weight of the composition.

The weight ratio of the (b) taurate surfactant(s) to the (c) amino acid surfactant(s) included in the composition may be from 3:1 to 1:10, preferably 2:1 to 1:8, and more preferably from 1:1 to 1:5.

) The total amount of the (a) glycolipid(s), the (b) taurate surfactant(s), and the (c) amino acid surfactant(s) included in the composition may be from 1% to 25% by weight, preferably from 3% to 20% by weight, and more preferably from 5% to 15% by weight, relative to the total weight of the composition.

The composition may further comprise at least one monovalent non-polymeric acid or a salt thereof preferably a monovalent non-polymeric carboxylic acid, and more preferably a monovalent hydroxylhydroxy acid such as lactic acid and salicylic acid.

The pH of the composition may be from 4 to 8, preferably 4.5 to 7.5, and more preferably from 5 to 7.

The composition may be a cleansing composition, preferably a cleansing composition for skin, and more preferably a cleansing composition for the face.

The composition may include at least one higher fatty acid in an amount of 1% by weight or less, preferably 0.5% by weight or less, and more preferably 0.3% by weight or less, relative to the total amount of the composition.

The present invention also relates to a non-therapeutic cosmetic process for caring for and/or cleansing keratinous substances, such as skin, comprising applying onto the keratinous substances the composition according to the present invention, and optionally removing the composition from the keratin substance.

### BEST MODE FOR CARRYING OUT THE INVENTION

After diligent research, the inventors have surprisingly discovered that a composition comprising (a) at least one glycolipid, (b) at least one taurate surfactant in a certain amount, and (c) at least one amino acid surfactant, can provide a stable composition with regard to a temperature change, thus achieving the objective of the present invention.

Thus, the composition according to the present invention is a composition comprising:
(a) at least one glycolipid;
(b) at least one taurate surfactant in an amount of at least 0.6% by weight relative to the total weight of the composition;
(c) at least one amino acid surfactant; and
(d) water.

Hereinafter, the composition, process, and use according to the present invention will be explained in a more detailed manner.

### [Composition]

The composition is preferably a cosmetic composition, and more preferably a non-therapeutic, cosmetic composition for a keratinous substance. The keratinous substance here means a material containing keratin as a main constituent element, and examples thereof include the skin, scalp, lips, and the like. In one preferred embodiment, the composition according to the present invention is a cosmetic composition for the skin. The composition according to the present invention may be intended for application onto a keratinous substance, preferably skin, and in particular facial skin.

In one embodiment of the present invention, the composition according to the present invention is a rinse-off type composition, and more preferably a rinse-off composition for caring for and/or cleansing keratinous substances. The rinse-off composition can be removed from a keratin substance such as skin, preferably with water.

The composition according to the present invention may more preferably be a rinse-off cleansing composition. The rinse-off cleansing composition can remove sebum and/or makeup on a keratin substance such as skin, and can be removed from the keratin substance, preferably with water.

The form of the composition according to the present invention is not particularly limited. In general, the composition according to the present invention is liquid at room temperature (25° C.) and atmospheric pressure (105 Pa). The composition may take various forms, such as a solution, an aqueous solution, a toner, a micellar water, a lotion, a milky lotion, a cream, a gel, a liquid gel, a paste, a serum, a suspension, a dispersion, a fluid, a milk, an emulsion (O/W or W/O form), or the like. It is preferable that the composition according to the present invention be in the form of an 35 aqueous solution or gel.

Preferably, the form of the present invention is an aqueous formula, such as an aqueous solution, a toner, a serum, or a micellar water.

40) The composition according to the present invention is stable under a variety of temperature conditions such as from very low temperature, for example −5° C., to room temperature (25° C.). In addition, the composition according to the present invention is stable under a wide range of temperature changes, for example, from −20° C. to 20° C. The term “stable” here means the composition maintains its original uniform or homogeneous aspect over time without causing a sedimentation.

The composition according to the present invention comprises (a) at least one glycolipid, (b) at least one taurate surfactant, (c) at least one amino acid surfactant, and (d) water. The ingredients in the composition will be described in a detailed manner below.

### (Glycolipid)

The composition according to the present invention comprises (a) at least one glycolipid. A single type of glycolipid may be used, or two or more different types of glycolipids may be used in combination.

The term “glycolipid”′ is understood as meaning a compound formed from a lipid to which are attached one or more sugar compounds.

The (a) glycolipids may be selected from rhamnolipids, sophorolipids, glucolipids, trehalolipids, cellobiose lipids and mixtures thereof.

The (a) glycolipids are preferably selected from rhamnolipids, sophorolipids and mixtures thereof, more preferably rhamnolipids.

### Glucolipids:

The (a) glycolipids may be glucolipids, which contain a glucose moiety and can be represented by the general formula (I):

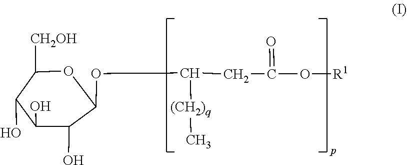

in which:
R¹ represents a hydrogen atom or a cation,
p denotes an integer ranging from 1 to 4, and
q denotes an integer ranging from 4 to 10, preferably equal to 6.

The glucolipids can be produced by the bacterium Alcaligenes sp. MM1.

Appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids

The (a) glycolipids may be sophorolipids, which contain a sophorose moiety and can be represented by the general formula (II):

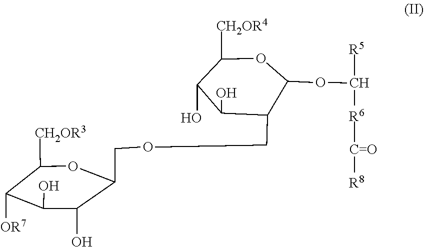

in which:
R³ and R⁴ individually represent a hydrogen atom or an acetyl group,
R⁵ represents a saturated or unsaturated, hydroxylated or non-hydroxylated hydrocarbon group having from 1 to 9 carbon atoms, preferably methyl,
R⁶ represents a saturated or unsaturated, hydroxylated or non-hydroxylated hydrocarbon group having from 1 to 19 carbon atoms, with the proviso that the total number of carbon atoms in the groups R⁵ and R⁶ does not exceed 20 and is preferably from 14 to 18.

Sophorolipids may be incorporated into the composition according to the present invention either in the form of an open-chain free acid, where R⁷ represents a hydrogen atom and R⁸ represents a hydroxy group OH, or in its lactone form, where a lactone ring is formed between R⁷ and R⁸, as indicated by formula (III):

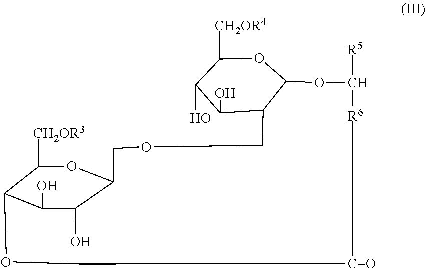

in which:
R³, R⁴, R⁵ and R⁶ are as defined above, with the proviso that at least one of R³ and R⁴ represents an acetyl group.

The sophorolipids can be produced by yeast cells, for example, Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

Use as a sophorolipid is possible, for example the product sold under the name Sopholiance S by Givaudan and the product sold under the name BioToLife by BASF.

### Trehalolipids

The (a) glycolipids may be trehalolipids, which contain a trehalose fragment and can be represented by the general formula (IV):

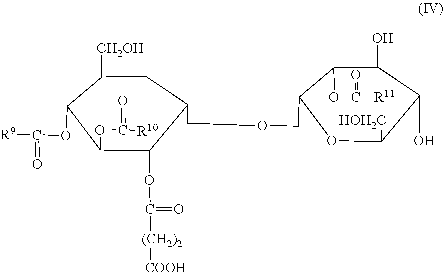

in which:
R⁹, R¹⁰ and R¹¹ individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids

The (a) glycolipids may be cellobiose lipids, which contain a cellobiose fragment and can be represented by the general formula (V):

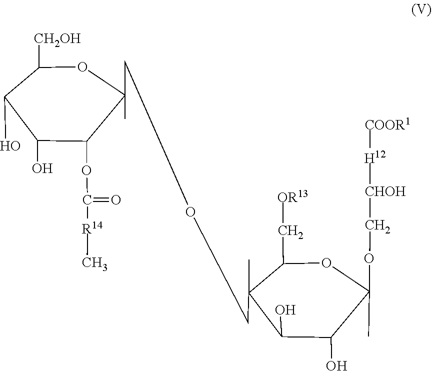

in which:
R¹ represents a hydrogen atom or a cation,
R¹² represents a saturated or unsaturated, hydroxylated or non-hydroxylated hydrocarbon radical having from 9 to 15 carbon atoms, preferably 13 carbon atoms,
R¹³ represents a hydrogen atom or an acetyl group; and
R¹⁴ represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The (a) glycolipids may be rhamnolipids.

The composition according to the present invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

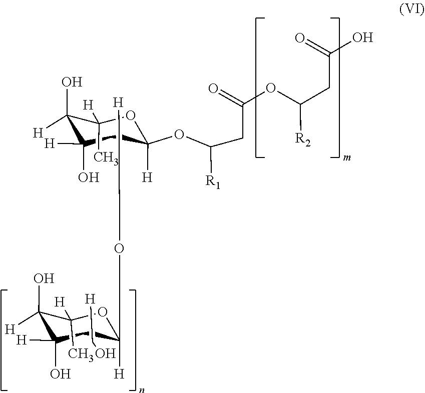

in which:
m denotes an integer equal to 2, 1 or 0,
n denotes an integer equal to 1 or 0, and
R¹ and R², each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the invention preferably comprises at least one di-rhamnolipid.

The composition according to the present invention preferably comprises at least one di-rhamnolipid of formula (VI) in which:
m denotes an integer equal to 2, 1 or 0;
n denotes an integer equal to 1; and
R¹ and R², each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the present invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C₁-C₂₀ alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the present invention preferably comprises at least one di-rhamnolipid of formula (VI) in which:
m denotes an integer equal to 2, 1 or 0;
n denotes an integer equal to 1; and
R¹ and R², which are identical or different, are selected from pentenyl, hexenyl, heptenyl, octenyl, nonenyl, decenyl, undecenyl, dodecenyl and tridecenyl radicals and radicals of formula-(CH₂)ₒCH₃, with o denoting an integer ranging from 1 to 23, in particular from 3 to 15 and more particularly from 4 to 12.

According to one embodiment of the invention, the composition according to the invention comprises at least one di-rhamnolipid of general formula (VI) in which m is equal to 1

According to one embodiment of the invention, the composition according to the invention comprises a mixture of at least two, preferably at least three, di-rhamnolipids of general formula (VI) in which m is preferably equal to 1.

According to another embodiment of the invention, the composition according to the invention comprises a mixture comprising at least one mono-rhamnolipid.

More preferably, the composition according to the invention comprises at least one dirhamnolipid of the following formula (VII):

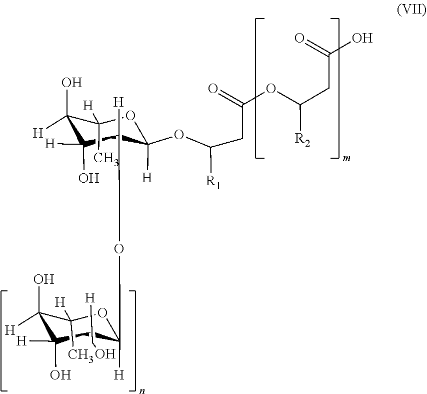

in which:
m denotes an integer equal to 2, 1 or 0; preferably, m is equal to 1,
n denotes an integer equal to 1,
R₁ is a —(CH₂)ₚ—CH₃ radical, with p being an integer varying from 1 to 23, preferably from 4 to 12,
R² is a —(CH₂)_{q}—CH₃ radical, with q being an integer varying from 1 to 23, preferably from 4 to 12, and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R₁ and R₂ respectively represented by the symbols CX and CY, the integers X and Y being respectively equal to p+4 and q+4.

TABLE

| Composes | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the invention in a proportion of at least 50% by weight and preferably of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R₁ represents a —(CH₂)OCH₃ radical, with o being an integer varying from 4 to 12, and R² is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

According to another preferred embodiment, the composition according to the present invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R₁ represents a —(CH₂)OCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

Preferably, the composition according to the present invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids, 5
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R₁ represents a —(CH₂)₆CH₃ radical and R² represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

The amount of the (a) glycolipid(s) in the composition according to the present invention may be from 0.5% by weight or more, preferably 1% by weight or more and more preferably 2% by weight or more, and/or may be 20% by weight or less, preferably 15% by weight or less, and more preferably 10% by weight or less, relative to the total weight of the composition. Any combinations of the values of the upper and lower limit are available.

The amount of the (a) glycolipid(s) in the composition according to the present invention may be from 0.5% to 20% by weight, preferably from 1% to 15% by weight, and more preferably from 2% to 10% by weight, relative to the total weight of the cosmetic composition.

In the context of the present specification, any combinations of the upper limit values and the lower limit values above can be available to represent the preferred range of the amount.

### (Taurate Surfactant)

The composition according to the present invention comprises (b) at least one taurate surfactant. A single type of the taurate surfactant may be used, but two or more different types of the taurate surfactants may be used in combination.

The term “taurate surfactant” here means an anionic surfactant comprising at least one taurate moiety. The taurate surfactant can be also called as a taurate-based surfactant.

The (b) taurate surfactant is preferably acyl taurate, more preferably N-acyl taurate, and even more preferably N-acyl methyl taurate (i.e. N-acyl-N-methyltaurate).

The (b) taurate surfactants include those of Formula I, below:

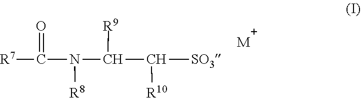

wherein,
R⁷ is (C₈-C₂₂) alkyl;
R⁸ is H or (C₁-C₄) alkyl;
R⁹ and R¹⁰ are each independently H or (C₁-C₄) alkyl; and M is a sodium, potassium, or ammonium cation.

The (b) taurate surfactant may be selected from the group consisting of taurate, caproyl taurate, lauroyl taurate, myristoyl taurate, palmitoyl taurate, stearoyl taurate, oleoyl taurate, cocoyl taurate, methyl taurate, coconut oil fatty acid methyl taurate, palm kernel oil fatty acid methyl taurate, hydrogenated palm kernel oil fatty acid methyl taurate, beef tallow fatty acid methyl taurate, hydrogenated beef tallow fatty acid methyl taurate, caproyl methyl taurate, lauroyl methyl taurate, myristoyl methyl taurate, palmitoyl methyl taurate, stearoyl methyl taurate, oleoyl methyl taurate, cocoyl methyl taurate, methyltaurine cocoyl methyl taurate, and salts thereof.

For example, the (b) taurate surfactants include sodium methyl lauroyl taurate, sodium methyl myristoyl taurate, potassium methyl myristoyl taurate, sodium methyl cocoyl taurate, sodium methyl oleoyl taurate, calcium methyl lauroyl taurate, potassium methyl lauroyl taurate, and ammonium methyl lauroyl taurate. Likewise, in some instances, the taurate surfactant is sodium methyl cocoyl taurate.

Examples of the (b) taurate surfactant include, but are not limited to:
sodium salt of palm kernel oil methyltaurate, sold under the name Hostapon CT Paté® by Clariant;
sodium N-cocoyl-N-methyltaurate, sold under the name Hostapon LT-SF® by Clariant or sold under the name Nikkol CMT-30-T® by Nikkol;
sodium methyl stearoyl taurate sold under the name Nikkol SMT®;
sodium palmitoyl methyltaurate, sold under the name Nikkol PMT® by Nikkol; and
sodium methyl cocoyl taurate, sold under the name PUREACT WS CONC LO by Innospec Active Chemicals.

The amount of the (b) taurate surfactant(s) in the composition according to the present invention is at least 0.6% by weight relative to the total weight of the composition. The amount of the taurate surfactant(s) in the composition may be 0.75% by weight or more, and preferably 0.9% by weight or more, relative to the total weight of the composition. The amount of the taurate surfactant(s) in the composition according to the present invention may be 10% by weight or less, preferably 5% by weight or less, and more preferably 3% by weight or less, relative to the total weight of the composition. Any combinations of the values of the upper and lower limit are available.

The amount of the (b) taurate surfactant(s) in the composition according to the present invention may be from 0.6% to 10% by weight, preferably from 0.75% to 5% by weight, and more preferably from 0.9% to 3% by weight, relative to the total weight of the composition.

### (Amino Acid Surfactant)

The composition according to the present invention comprises (c) at least one amino acid surfactant. A single type of the amino acid surfactant may be used, but two or more different types of the amino acid surfactants may be used in combination.

The term “amino acid surfactant” here means an anionic surfactant based on amino acids or derivatives thereof. Typically, the amino acid surfactant is an anionic surfactant comprising at least one amino moiety and at least one carboxylic acid moiety which is in the form of a carboxylate. The amino acid surfactant may have two or more of amino moieties and/or two or more carboxylic acid moieties which are in the form of carboxylates. The amino acid surfactant can be also called as an amino acid-based surfactant.

The (c) amino acid surfactant is different from the (b) taurate surfactant here.

The (c) amino acid surfactant may preferably be selected from amino acid derivatives. The amino acid derivative may more preferably be selected from salts of amino acids and N-acylated amino acids, for example alkali metal salts and alkali earth metal salts of amino acids and N-acylated amino acids, such as sodium salts, potassium salts, magnesium salts, and calcium salts of amino acids and N-acylated amino acids. Thus, the (c) amino acid surfactant is preferably a N-acyl amino acid surfactant.

The acyl group which forms the N-acyl moiety of the amino acid derivatives may be a C₁-C₃₀ acyl group, preferably a C₆-C₂₈ acyl group, and more preferably a C₁₂-C₂₄ acyl group.

The (c) amino acid surfactant may even more preferably be selected from the group consisting of glutamates, N-acylated glutamates, aspartates, N-acylated aspartates, and salts thereof.

The carboxylate salts of these amino acids can be formed by conventional means such as by neutralization of the respective amino acid with a base. The amine group situated on the α-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈ to C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

In one embodiment, said amino acid based anionic surfactant is represented by the formula (A):

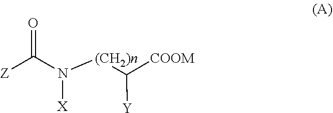

wherein:
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)OM, and
M is a salt-forming cation wherein COO is the counter-anion, such as for example sodium, potassium, ammonium, or triethanolamine.

According to a preferred embodiment of the present invention, in the amino fatty acid of formula (A):
Z represents a saturated or unsaturated, linear C₈ to C₁₈ alkyl group, in particular a cocoyl group,
X is hydrogen,
n is 0,
Y is hydrogen, and
M is a salt-forming cation wherein COO is the counter-anion, such as for example sodium, potassium, ammonium, or triethanolamine.

Examples of the amino acid surfactants are salts of alanine, arginine, aspartic acid, glutamic acid, glycine, isoleucine, leucine, lysine, phenylalanine, serine, tyrosine, valine, sarcosine, and any mixture thereof. More specifically, mention can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium 35 cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, 40) disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

Reference can be made to the commercially available amino acid surfactants of, for example:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate MN® by Nikkol, or sodium palmitoyl sarcosinate, sold under the name Nikkol Sarcosinate PN® by Nikkol;
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken;
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto; disodium glutamates, disodium stearoyl glutamate sold under the name Amisoft® HS-21P by Ajinomoto, and mixtures thereof; sodium cocoyl glutamate, sold under the name Plantapon® Amino SF-N by BASF Japan; disodium cocoyl glutamate, sold under the name Plantapon® Amino SCG-L by BASF Japan; and disodium/sodium cocoyl glutamate, sold under the name Amisoft® CS-22 by Ajinomoto;
aspartates, such as the mixture of triethanolamine N-lauroyl aspartate and triethanolamine N-myristoyl aspartate, sold under the name Asparack® by Mitsubishi; and
glycine derivatives (glycinates), such as sodium N-cocoyl glycinate, sold under the names Amilite GCS-12® and Amilite GCK 12 by Ajinomoto.

The amount of the (c) amino acid surfactant(s) in the composition may be 0.5% by weight or more, preferably 1% by weight or more, and preferably 2% by weight or more, relative to the total weight of the composition. The amount of the (c) amino acid surfactant(s) in the composition according to the present invention may be 20% by weight or less, preferably 15% by weight or less, and more preferably 10% by weight or less, relative to the total weight of the composition. Any combinations of the values of the upper and lower limit are available.

The amount of the (c) amino acid surfactant(s) in the composition according to the present invention may be from 0.5% to 20% by weight, preferably from 1% to 15% by weight, and more preferably from 2% to 10% by weight, relative to the total weight of the composition.

In one embodiment of the present invention, the weight ratio of the (b) taurate surfactant(s) to the: (c) amino acid surfactant(s) included in the composition may be from 3:1 to 1:10, preferably 2:1 to 1:8, and more preferably from 1:1 to 1:5.

In another embodiment of the present invention, the total amount of the (b) taurate surfactant(s) and the (c) amino acid surfactant(s) included in the composition may be from 1% to 20% by weight, preferably from 2% to 15% by weight, and more preferably from 3% to 10% by weight, relative to the total weight of the composition.

In another embodiment of the present invention, the weight ratio of the (a) glycolipid(s) to the total amount of the (b) taurate surfactant(s) and the (c) amino acid surfactant(s) included in the composition may be from 10:1 to 1:10, preferably 5:1 to 1:5, and more preferably from 3:1 to 1:3, and in particular from 2:1 to 1:2.

In another embodiment of the present invention, the total weight amount of the (a) glycolipid(s), the (b) taurate surfactant(s), and the (c) amino acid surfactant(s) included in the composition may be from 1% to 25% by weight, preferably from 3% to 20% by weight, and more preferably from 5% to 15% by weight, relative to the total weight of the composition.

### (Water)

The composition according to the present invention includes water.

The amount of the water in the composition according to the present invention may be 50% by weight or more, preferably 60% by weight or more, and more preferably 70% by weight or more, relative to the total weight of the composition.

The amount of the water in the composition according to the present invention may be 99% by weight or less, preferably 97% by weight or less, and more preferably 95% by weight or less, relative to the total weight of the composition.

The amount of the water in the composition according to the present invention may be from 50% to 99% by weight, preferably from 60% to 97% by weight, and more preferably from 70% to 95% by weight, relative to the total weight of the composition.

### (Other Optional Ingredients)

### Polyol

The composition according to the present invention may or may not comprise at least one polyol. Two or more polyols may be used in combination. Thus, a single type of the polyol or a combination of different types of the polyols may be used. 25

For the purposes of the present invention, the term “polyol” should be understood as meaning any organic molecule comprising at least two free hydroxyl groups.

The polyol that is suitable for use in the invention may be a compound of linear, branched or cyclic, saturated or unsaturated alkyl type, bearing at least two-OH functions on the alkyl chain.

Preferably, a polyol that may be used in the composition according to the invention is a compound of linear or branched, preferably linear alkyl type bearing at least two-OH functions, preferably 2 to 5-OH functions, more preferably 2 to 4-OH functions, and even more preferably 2 or 3-OH functions on the alkyl chain.

The polyols that are advantageously suitable for formulating the cosmetic compositions according to the present invention are those especially having from 2 to 12 carbon atoms or for example, 3 to 8 carbon atoms.

The polyols that may be used according to the present invention are chosen from linear or branched, preferably linear polyols having from 3 to 8 carbon atoms; mention may be made especially of:
diols such as hexylene glycol, dipropylene glycol, pentylene glycol, propylene glycol, butylene glycol, and caprylyl glycol; and
triols, such as glycerol (glycerin), and mixtures thereof.

The amount of the polyol(s) in the composition according to the present invention may be from 1% by weight or more, preferably 2% by weight or more, and more preferably 3% by weight or more, and/or may be 30% by weight or less, preferably 25% by weight or less, and more preferably 20% by weight or less, relative to the total weight of the composition.

The amount of the polyol(s) in the composition according to the present invention may be from 1% to 30% by weight, preferably from 2% to 25% by weigh, and more preferably from 3% to 20% by weight, relative to the total weight of the composition.

### Monovalent Non-Polymeric Acid or Salt Thereof

The composition according to the present invention may or may not comprise at least one monovalent non-polymeric acid or a salt thereof. A single type of monovalent non-polymeric acid or a salt thereof be used, or two or more different types of monovalent non-polymeric acids or salts thereof may be used in combination . . .

The monovalent non-polymeric acid or a salt thereof is different from the amino acid surfactant and the taurate surfactant.

The term “non-polymeric” here means that the acid is not obtained by polymerizing two or more monomers. Therefore, the non-polymeric acid does not correspond to an acid obtained by polymerizing two or more monomers such as polyacrylic acids. 20) The term “salt” here means a salt formed by addition of suitable base(s) to the monovalent non-polymeric acid, which may be obtained from a reaction with the monovalent non-polymeric acid with the base(s) according to methods known to those skilled in the art. As the salt, mention May be made of metal salts, for example salts with alkaline metal such as Na and K, and salts with alkaline earth metal such as Mg and Ca, and ammonium salts.

It is preferable that the molecular weight of the monovalent non-polymeric acid or salt thereof be less than 1,000, preferably 500 or less, and more preferably 200 or less.

The monovalent non-polymeric acid or a salt thereof can be included in the aqueous phase formed by the (d) water.

The monovalent non-polymeric acid has a single acid group which may be selected from the group consisting of a carboxylic group, a sulfuric group, a sulfonic group, a phosphoric group, a phosphonic group, and a mixture thereof.

The monovalent non-polymeric acid or a salt thereof may be selected from monovalent organic or inorganic acids and salts thereof.

It is preferable that the monovalent non-polymeric acid be a monovalent organic acid, and more preferably a monovalent non-polymeric carboxylic acid.

The monovalent non-polymeric carboxylic acid may be selected from hydroxyl acids, and preferably alpha-hydroxy acids and beta-hydroxy acids. As the alpha-hydroxy acids, mention may be made of, for example, lactic acid and glycolic acid. As the beta-hydroxy acids, mention may be made of, for example, salicylic acid.

Thus, the monovalent non-polymeric acid may be a monovalent non-polymeric organic acid, 50 preferably a monovalent non-polymeric carboxylic acid, and more preferably a monovalent hydroxyl acid such as lactic acid and salicylic acid.

The amount of the monovalent non-polymeric acid(s) or salt(s) thereof in the composition according to the present invention may be 0.01% by weight or more, preferably 0.05% by weight or more, and more preferably 0.1% by weight or more, relative to the total weight of the composition.

The amount of the monovalent non-polymeric acid(s) or salt(s) thereof in the composition according to the present invention may be 10% by weight or less, preferably 5% by weight or less, and more preferably 3% by weight or less, relative to the total weight of the composition.

The amount of the monovalent non-polymeric acid(s) or salt(s) thereof in the composition according to the present invention may be from 0.01% to 10% by weight, preferably from 0.05% to 5% by weight, and more preferably from 0.1% to 3% by weight, relative to the total weight of the composition.

### Non-Polymeric Acids Having Two or More Acid Dissociation Constants

The composition according to the present invention may or may not include at least one non-polymeric acid having two or more pKa values or a salt thereof. A single type of non-polymeric acid having two or more pKa values or a salt thereof may be used, or two or more different types of non-polymeric acids having two or more pKa values or salts thereof may be used in combination.

The non-polymeric acid having two or more pKa values or the salt thereof has two or more acid dissociation constants or salt(s) thereof. The pKa value (acid dissociation constant) is well known to those skilled in the art, and should be determined at a constant temperature such as 25° C.

The composition according to the present invention may include two or more non-polymeric acids having two or more pKa values or salt(s) thereof in combination.

The term “non-polymeric” here means that the acid is not obtained by polymerizing two or more monomers. Therefore, the non-polymeric acid does not correspond to an acid obtained by polymerizing two or more monomers such as polycarboxylic acid. 35 It is preferable that the molecular weight of the non-polymeric acid having two or more pKa values or salt(s) thereof is 1,000 or less, preferably 800 or less, and more preferably 700 or less.

There is no limit to the type of the non-polymeric acid having two or more pKa values or salt(s) thereof. Two or more different types of non-polymeric acids having two or more pKa values or salts thereof may be used in combination. Thus, a single type of a non-polymeric acid having two or more pKa values or a salt thereof or a combination of different types of non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metal such as Na and K, and salts with alkaline earth metal such as Mg and Ca, and ammonium salts.

The non-polymeric acid having two or more pKa values or salt(s) thereof may be an organic acid or salt(s) thereof, and preferably a hydrophilic or water-soluble organic acid or salt(s) thereof.

The non-polymeric acid having two or more pKa values may have at least two acid groups selected from the group consisting of a carboxylic group, a sulfuric group, a sulfonic group, a phosphoric group, a phosphonic group, a phenolic hydroxyl group, and a mixture thereof.

The non-polymeric acid having two or more pKa values may be a non-polymeric polyvalent acid.

The non-polymeric acid having two or more pKa values may be selected from the group consisting of dicarboxylic acids, disulfonic acids, and diphosphoric acids, and a mixture thereof.

The non-polymeric acid having two or more pKa values or salt(s) thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, lactic acid, phosphoric acid, aconitic acid, oxaloacetic acid, tartaric acid, and salts thereof; aspartic acid, glutamic acid, and salts thereof; terephthalylidene dicamphor sulfonic acid or salts thereof (Mexoryl SX), Benzophenone-9; phytic acid, and salts thereof; and salts thereof; Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Conc), Green 205 (Light Green SF 25 Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphthol Green B), Green 402 (Guinea Green B), and Black 401 (Naphthol Blue Black); folic acid, ascorbic acid, erythorbic acid, and salts thereof; cystine and salts thereof; EDTA and salts thereof; glycyrrhizin and salts thereof; and a mixture thereof.

It may be preferable that the non-polymeric acid having two or more pKa values or salt(s) thereof be selected from the group consisting of lactic acid, phosphoric acid, phytic acid, and salts thereof.

The amount of the non-polymeric acid(s) having two or more pKa values or salt(s) thereof in the composition according to the present invention may be 0.01% by weight or more, preferably 0.05% by weight or more, and more preferably 0.1% by weight or more, relative to the total weight of the composition.

The amount of the non-polymeric acid(s) having two or more pKa values or salt(s) thereof in the composition according to the present invention may be 10% by weight or less, preferably 5% by weight or less, and more preferably 3% by weight or less, relative to the total weight of the composition.

The amount of the non-polymeric acid(s) having two or more pKa values or salt(s) thereof in the composition according to the present invention may be from 0.01% to 10% by weight, preferably from 0.05% to 5% by weight, and more preferably from 0.1% to 3% by weight, relative to the total weight of the composition.

### Higher Fatty Acid

The composition according to the present invention may or may not comprise at least one higher fatty acid. A single type of higher fatty acid may be used, or two or more different types of higher fatty acids may be used in combination

The term “fatty acid” here means a monofunctional carboxylic acid with an aliphatic chain.

The higher fatty acid may be linear or branched. The higher fatty acid is preferably selected from linear higher fatty acids.

The higher fatty acid may be saturated or unsaturated. In one preferred embodiment of the present invention, the fatty acid is selected from saturated fatty acids. In another preferred embodiment of the present invention, the fatty acid is selected from unsaturated fatty acids having 1 to 3 carbon-carbon double bonds, and preferably 1 or 2 carbon-carbon double bonds.

Non-limiting examples of the higher fatty acid include fatty acids having from 6 to 24 carbon atoms, preferably from 8 to 20 carbon atoms.

In a preferred embodiment of the present invention, the higher fatty acid is a mixture of higher fatty acids selected from linear fatty acids having from 6 to 24 carbon atoms, and preferably from 8 to 20 carbon atoms, wherein the fatty acids are saturated or unsaturated with 1 or 2 carbon-carbon double bonds.

The higher fatty acid can be represented by the following formula:

RCOOH

wherein:

R is a linear or branched, preferably linear, C₆-C₂₄ alkyl or alkenyl group, preferably a C₈-C₂₀ alkyl or alkenyl group. R may comprise 1 or 2 carbon-carbon double bonds.

As non-limiting examples of the higher fatty acids, mention can be made of capric acid, caprylic acid, lauric acid, linoleic acid, myristic acid, oleic acid, palmitic acid, stearic acid, and mixtures thereof.

The higher fatty acid(s) may be present in an amount of 0.01% by weight or more, preferably 0.05% by weight or more, more preferably 0.08% by weight or more, and/or may be present in an amount of 5% by weight or less, preferably 3% by weight or less, more preferably 1% by weight or less, relative to the total weight of the composition.

The amount of the active agent(s) for chemical peels in the composition according to the present invention may be from 0.01% to 5% by weight, preferably from 0.05% to 3% by weigh, and more preferably from 0.08% to 1% by weight, relative to the total weight of the composition.

In another embodiment of the present invention, the composition according to the present invention comprises fatty acids or the higher fatty acids in an amount of 1% by weight or less, preferably 0.5% by weight or less, and more preferably 0.3% by weight or less, relative to the total amount of the composition.

### pH Adjusting Agent

The pH of the composition according to the present invention may may not be adjusted to the desired value using at least one pH adjusting agent, such as an acidifying or a basifying agent, for example, which are commonly used in cosmetic products.

The pH of the composition according to the present invention may be from 4 to 8, preferably 4.5 to 7.5, and more preferably from 5 to 7.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide; quaternary ammonium hydroxides and guanidinium hydroxide; alkali metal silicates, such as sodium metasilicates; amino acids, preferably basic amino acids, such as arginine, lysine, ornithine, citrulline and histidine; carbonates and bicarbonates, particularly of a primary amine, secondary amine or tertiary amine, of an alkali metal or alkaline-earth metal, or of ammonium; and the compounds of the following formula:

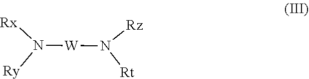

in which
W is a C₁-C₆ alkylene residue optionally substituted with a hydroxyl group or a C₁-C₆ alkyl group;
Rx, Ry, Rz and Rt, which may be identical or different, represent a hydrogen atom or a C₁-C₆ alkyl,
C₁-C₆ hydroxyalkyl or C₁-C₆ aminoalkyl group. Mention may especially be made of 1,3-diaminopropane, 1,3-diamino-2-propanol, spermine and spermidine.

The pH adjusting agent(s) may be used in an amount ranging from 0.001% to 10% by weight, preferably from 0.01% to 5% by weight, and more preferably from 0.1% to 3% by weight, relative to the total weight of the composition.

The pH value of the composition according to the present invention is not particularly limited, however, in general it ranges from 3 to 8, preferably from 3.5 to 7, and more preferably from 4 to 6.

### Additives

The composition according to the present invention may or may not comprise any optional additive(s) usually used in the field of cosmetics, chosen, for example, from anionic, cationic, nonionic or amphoteric polymers, such as polylysine, anionic, cationic, nonionic or amphoteric surfactants other than the ingredients (a) to (c), oils, hydrophobic organic solvents, gums, resins, thickeners, dispersants, antioxidants, film-forming agents, buffers, preserving agents, such as sodium benzoate, fragrances, neutralizers, antiseptics, chelating agents, UV-screening agents, cosmetic active agents such as vitamins, moisturizers, emollients or collagen-protecting agents, and mixtures thereof.

Polylysines are cationic polymers and correspond to the condensation of several amino acids of lysine. Polylysine can be a natural homopolymer of L-lysine that can be produced by bacterial fermentation. Polylysines are typically used as a natural preservative in food products. Polylysine is a polyelectrolyte which is soluble in polar solvents such as water.

Polylysine can be, for example, epsilon-polylysine (or referred as “ε-polylysine”), which is a condensation of amino groups at the-position and carboxyl groups of lysines, or alpha-polylysine (or referred as “α-polylysine”), which is a condensation of amino groups at the α-position and carboxyl groups of lysines. Polylysine is commercially available in various forms, such as poly D-lysine and poly L-lysine. The polylysine is generally a condensate of L-lysines, 10) i.e., poly L-lysine.

The molecular weight (Da) of the polylysine may be more than 1,000 and less than 20,000, preferably more than 1,500 and less than 15,000, and more preferably more than 2,000 and less than 10,000.

The composition according to the present invention may comprise the above optional ingredient(s) in an amount of from 0.01% to 30% by weight, preferably from 0.05% to 20% by weight, and more preferably from 0.1% to 10% by weight, relative to the total weight of the composition.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

The composition according to the present invention can be prepared by simple or easy mixing with a conventional mixing means such as a stirrer and a homogenizer. Also, heating may not be necessary. Therefore, the process for preparing the composition according to the present invention may be environmentally friendly.

### [Cosmetic Application]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition, and more preferably a face care composition.

In particular, the composition according to the present invention is useful for cleansing. Thus, it is preferable that the composition according to the present invention be a cleansing composition, more preferably a cleansing composition for skin, and even more preferably a cleansing composition for face.

### [Cosmetic Process and Use]

The present invention also relates to a non-therapeutic method or process, preferably a cosmetic method or process, and more preferably a cosmetic method or process for caring for and/or cleansing keratinous substances, such as the skin, scalp, and lips, in particular facial skin, comprising:

applying onto the keratinous substance a composition comprising:
(a) at least one glycolipid;
(b) at least one taurate surfactant in an amount of at least 0.6% by weight relative to the total weight of the composition;
(c) at least one amino acid surfactant; and
(d) water.

The present invention also relates to:
a cosmetic process for a keratin substance such as skin, comprising applying to the keratin substance the composition according to the present invention; and optionally removing the composition from the keratin substance, or
a use of the composition according to the present invention for caring for and/or cleansing a keratin substance such as skin.

The cosmetic process here means a non-therapeutic cosmetic method, such as for a cosmetic method for caring for or cleansing the surface of a keratin substance such as skin. 25 The step of removing in the cosmetic process according to the present invention can be performed by, for example, rinsing off with water the composition according to the present invention from a keratin substance such as skin.

If the step of removing is not performed, the cosmetic process according to the present invention can be useful for caring for a keratin substance such as skin, because the (a) glycolipids can provide the keratin substance with anti-inflammation, anti-allergic, or anti-bacterial properties, which would be useful for, in particular, caring for skin with acne and sensitive skin.

If the step of removing is performed, the cosmetic process according to the present invention can be useful for cleansing a keratin substance such as skin, because the (a) glycolipids can function as a surfactant, as well as the (b) taurate surfactant and the (c) amino acid surfactant.

The composition is generally applied on a keratinous substance, such as skin, with the hands or an applicator. The present invention may comprise an optional step of rinsing the composition from the keratinous substance after it is applied.

The same explanations for the composition, (a) glycolipid, the (b) taurate surfactant, the (c) amino acid surfactant, and (d) water as the composition according to the present invention above can be applied to the method, process, and use inventions. The composition used in the process and use according to the present invention may include any of the optional ingredients explained above for the composition according to the present invention.

### EXAMPLES

The present invention will be described in more detail by way of examples which however should not be construed as limiting the scope of the present invention.

### Examples 1 to 5 and Comparative Example 1 to 3

Homogeneous solution compositions according to Examples 1 to 5 and Comparative Examples 1 to 3 were prepared by mixing the ingredients as listed in Tables 1 and 2 with a magnetic stirrer. The numerical values for the amounts of the ingredients shown in the tables are all based on “% by weight” as active raw materials. As a glycolipid, rhamnolipid-based surfactant was used which was obtained from EVONIK (product name: RHEANCE® One).

### [Evaluation]

The stability of the each of compositions according to Examples 1 to 5 and Comparative Examples 1 to 3 was evaluated as follows.

(Cycle of +20° C./−20° C.)

Each of the compositions was placed in a cylindrical transparent sample vial (SV-30) and stored in a cycle incubator, where temperature is controlled in a cycle of +20° C. and −20° C. for 5 days. The appearance of the compositions was evaluated by observing with naked eyes, and was evaluated based on the following criteria.
Good: No precipitation was observed and maintained its original appearance.
Bad: White precipitation particles observed at the bottom of the sample vial.
Very Bad: White sedimentation observed at the bottom of the sample vial.

The temperature cycle consisted of the following steps:
(a) maintaining the sample at +20° C. for 6 hours,
(b) cooling the sample to −20° C. for 6 hours,
(c) maintaining the sample at −20° C. for 6 hours,
(d) heating the sample to +20° C. for 6 hours, and
(e) repeating the (a) step as above.
(−5° C., 4° C., or RT)

Each of the compositions was placed in a cylindrical transparent sample vial (SV-30) just after the preparation of the compositions and stored in an incubator at −5° C., 4° C., or at room temperature for 7 days. The appearance of the compositions was evaluated by observing with naked eyes, and was evaluated based on the following criteria.
Good: No precipitation was observed and maintained its original appearance.
Bad: White precipitation particles observed at the bottom of the sample vial.
Very Bad: White sedimentation observed at the bottom of the sample vial.

The results are summarized in Tables 1 and 2 below.

TABLE 1

| | | | | Comp | Comp | Comp |
|---|---|---|---|---|---|---|
| Ingredient | Ex. 1 | Ex. 2 | Ex. 3 | Ex. 1 | Ex. 2 | Ex. 3 |
| Salicylic Acid | 0.2 | 0.2 | 0.2 | 0.2 | 0.2 | 0.2 |
| Sodium Citrate Dihydrate | 1.1 | 1.1 | 1.1 | 1.1 | 1.1 | 1.1 |
| Sodium Methyl Cocoyl Taurate | 0.6 | 0.9 | 1 | 0.2 | 0.4 | 0.5 |
| Disodium Cocoyl Glutamate | 3.6 | 3.3 | 3.2 | 3.9 | 3.7 | 3.7 |
| Sodium Cocoyl Glutamate | 0.84 | 0.79 | 0.77 | 0.92 | 0.88 | 0.86 |
| Rhamnolipid | 4.5 | 4.5 | 4.5 | 4.5 | 4.5 | 4.5 |
| Sodium Chloride | 0.11 | 0.21 | 0.23 | 0.047 | 0.093 | 0.12 |
| Coconut Acid | 0.05 | 0.09 | 0.1 | 0.02 | 0.04 | 0.05 |
| Citric Acid | 1.2 | 1.5 | 1.15 | 1.3 | 1.2 | 1.2 |
| Sodium Benzoate | 0.15 | 0.15 | 0.15 | 0.15 | 0.15 | 0.15 |
| Water | qs 100 | qs 100 | qs 100 | qs 100 | qs 100 | qs 100 |
| Evaluation | | | | | | |
| pH | 5.5 | 5.5 | 5.5 | 5.5 | 5.5 | 5.5 |
| Stability under cycle | Good | Good | Good | Very | Very | Very |
| (+20° C./−20° C.) | | | | Bad | Bad | Bad |
| Stability at −5° C. | Good | Good | Good | Bad | Good | Good |
| Stability at 4° C. | Good | Good | Good | Bad | Good | Good |
| Stability at RT | Good | Good | Good | Good | Good | Good |

TABLE 2

| Ingredient | Ex. 4 | Ex. 5 | Ex.6 |
|---|---|---|---|
| Glycerin | 5 | 5 | 5 |
| Rhamnolipid | 4.5 | 3.6 | 3.32 |
| Sodium Chloride | 0.58 | 2.4 | 3.76 |
| Disodium Cocoyl Glutamate | 2.0 | 1.8 | 1.8 |
| Sodium Methyl Cocoyl Taurate | 2.5 | 1.8 | 1.9 |
| Sodium Cocoyl Glutamate | 0.48 | 0.43 | 0.43 |
| Caprylyl Glycol | 0.8 | 0.6 | 0.6 |
| Salicylic Acid | 0.3 | 0.45 | 0.2 |
| Polylysine | 0.3 | 0.24 | 0.25 |
| Coconut Acid | 0.25 | 0.18 | 0.18 |
| Sodium Benzoate | 0.15 | 0.12 | 0.11 |
| Phytic Acid | 0.41 | — | — |
| Citric Acid | | | 0.07 |
| Water | qs 100 | qs 100 | qs 100 |

Evaluation

| | | | |
|---|---|---|---|
| pH | 5.5 | 5.5 | 5.5 |
| Stability under cycle | Good | Good | Good |
| (+20° C. /−20° C.) | | | |
| Stability at −5° C. | Good | Good | Good |
| Stability at 4° C. | Good | Good | Good |
| Stability at RT | Good | Good | Good |

As can be seen from Tables 1 and 2, the compositions according to Examples 1 to 5, which include the specific combination of the ingredients of the (a) glycolipid and the (b) taurate surfactant in a certain amount, as well as the (c) amino acid surfactant, showed an improved 5 stability against a temperature change as well as at a low temperature to RT.

On the other hand, the compositions according to Comparative Examples 1 to 3, which do not include the (b) taurate surfactant in an enough amount could not show an improved stability.

Accordingly, it can be said that the composition according to the present invention is very suitable as a cosmetic composition for caring for and/or cleansing keratinous substances, since it can provide a bioactive benefit produced from glycolipids with keratin substance while it exhibits improved stability.

1. A cosmetic composition comprising:
(a) at least one glycolipid;
(b) at least one taurate surfactant in an amount of at least 0.6% by weight relative to the total weight of the composition;
(c) (d) at least one amino acid surfactant; and water.

2. The composition according to claim 1, wherein the (a) glycolipid is selected from rhamnolipids.

3. The composition according to claim 1, wherein the amount of the (a) glycolipid(s) ranges from 0.5% to 20% by weight relative to the total weight of the cosmetic composition.

4. The composition according to claim 1, wherein the (b) taurate surfactant is represented by formula (I): 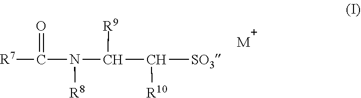
wherein
R⁷ is (C₈-C₂₂) alkyl;
R⁸ is H or (C₁-C₄) alkyl;
R⁹ and R¹⁰ are each independently H or (C₁-C₄) alkyl; and
M⁺ is a sodium, potassium, or ammonium cation.

5. The composition according to according to claim 1, wherein the amount of the (b) taurate surfactant(s) ranges from 0.6% to 10% by weight relative to the total weight of the composition.

6. The composition according to claim 1, wherein the (c) amino acid surfactant is represented by formula (A): 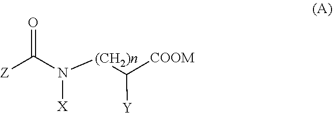
wherein:
Z represents a saturated or unsaturated, linear or branched hydrocarbon group having 8 to
carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —
CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and
M is a salt-forming cation wherein COO is the counter-anion.

7. The composition according to claim 1, wherein the amount of the (c) amino acid surfactant(s) ranges from 0.5% to 20% by weight relative to the total weight of the composition.

8. The composition according to claim 1, wherein the amount of the (d) water ranges from 50% to 99% by weight relative to the total weight of the composition.

9. The composition according to claim 1, wherein the weight ratio of the (b) taurate surfactant(s) to the (c) amino acid surfactant(s) included in the composition is from 3:1 to 1:10.

10. The composition according to claim 1, wherein the total amount of the (a) glycolipid(s), the (b) taurate surfactant(s), and the (c) amino acid surfactant(s) included in the composition is from 1% to 25% by weight relative to the total weight of the composition.

11. The composition according to claim 1, wherein the composition further comprises at least one monovalent non-polymeric acid or a salt thereof.

12. The composition according to claim 1, wherein the pH of the composition is from 4 to 8.

13. The composition according to claim 1, which is a cleansing composition.

14. The composition according to claim 1, wherein the composition includes at least one higher fatty acid in an amount of 1% by weight or less relative to the total amount of the composition.

15. A non-therapeutic cosmetic process for caring for and/or cleansing keratinous substances, comprising:
applying to the keratin substance the composition according to claim 1; and
optionally removing the composition from the keratin substance.
